(19) United States
(12) Patent Application Publication
(10) Pub. No.: US 2026/0338201 A1
(43) Pub. Date: Oct. 29, 2026

(54) COMPRESSOR AND AIR PURIFIER

(71) Applicant: Dyson Technology Limited, Malmesbury, Wiltshire (GB)

(72) Inventor: Thiago Rubens VIEIRA EBEL, Chippenham (GB)

(21) Appl. No.: 19/104,819

(22) PCT Filed: Aug. 16, 2023
(86) PCT No.: PCT/IB2023/058228

§ 371 Date: Feb. 19, 2025

(30) Foreign Application Priority Data
Aug. 31, 2022 (GB) 2212646.0

(51) Int. Cl. F04D 29/70; A61M 16/00; A61M 16/10; B01D 46/00; F04D 17/10; F04D 29/28; F04D 29/42

(52) U.S. Cl. CPC F04D 29/701; A61M 16/021; A61M 16/107; B01D 46/0047; F04D 17/10; F04D 29/284; F04D 29/4206; B01D 2273/30

(57) ABSTRACT

A compressor for an air purifier is provided and can include an impeller, a plurality of outlets, and a volute extending circumferentially around the compressor. The volute can pass air from the impeller to each of the plurality of outlets.

### FIELD OF THE INVENTION

The present invention relates to a compressor for an air purifier, in particular for a head-wearable air purifier, and to an air purifier comprising such a compressor.

### BACKGROUND OF THE INVENTION

Air purifiers typically move air through filters and/or nozzles to provide a filtered airflow. Head-wearable air purifiers have been developed which have nozzles for directing a filtered airflow to a user's face.

### SUMMARY OF THE INVENTION

A first aspect of the present invention provides a compressor for an air purifier, the compressor comprising: an impeller; a plurality of outlets; and a volute extending circumferentially around the compressor, the volute configured to pass air from the impeller to each of the plurality of outlets.

In this way, plural streams of air may be expelled from the compressor through the respective plurality of outlets. This may provide a more versatile compressor than, for example, a compressor comprising a volute configured to pass air to a single outlet. The compressor may also provide a more compact and convenient way of delivering air to the at least two outlets compared to, for example, two compressors each comprising a volute configured to pass air to a single outlet. The impeller may be larger than a similar impeller configured to provide air to a single outlet. In this way, the impeller may be able to provide the same, or a similar, flow rate and/or pressure to each outlet of the plurality of outlets as a smaller impeller could provide to a single outlet. The impeller may provide such flow rates and/or pressures while being operated at a lower speed than a smaller impeller that is configured to pass air to a single outlet. This may reduce a level of vibration and/or a level of noise of the compressor and/or an air purifier comprising the compressor. This may also improve an efficiency of the compressor, leading to reduced power draw and possibly increased battery life of an air purifier comprising the compressor.

Moreover, a compressor comprising a larger impeller may have larger relative tolerances compared to tolerances for a compressor having a smaller impeller. This may further reduce a noise of the compressor, as well as improving an efficiency and robustness of the compressor.

Optionally, the compressor is for a wearable air purifier, such as a head-wearable air purifier. Optionally, the compressor is for a room purifier or a vehicle cabin purifier. In any case, it will be understood from the above and following discussions that the compressor of the present invention may provide improved flexibility in terms of a location and/or configuration of the compressor on such a purifier, and may reduce a weight, vibration, noise and/or power draw of such a purifier, which may be particularly advantageous in a head-wearable purifier comprising the compressor.

Optionally, the plurality of outlets are circumferentially spaced around the compressor. In this way, the compressor may supply compressed air in different directions therefrom. Moreover, a number, or extent, of redirections of the airflow generated by the compressor, such as through ducting or channels of an air purifier comprising the compressor, may be reduced. This may particularly be the case when passing the airflow to different, particularly opposing, parts of such an air purifier. The compressor may thereby be more versatile than, for example, compressor having a single outlet.

Optionally, the plurality of outlets are equidistantly spaced around a circumference of the compressor. By spacing the outlets equidistantly, a cyclic load on the impeller, and/or on bearings supporting rotation of the impeller, may be reduced, such as by improving a symmetry of pressures and/or loads on the impeller.

Optionally, each outlet of the plurality of outlets is located on an opposite side of the compressor to another outlet of the plurality of outlets. This may reduce a cyclic load on the impeller, and/or on bearings supporting rotation of the impeller, such as by improving a symmetry of pressures and/or loads on the impeller on either side of a plane which includes an axis of rotation of the impeller. This may, in turn, reduce a vibration of the compressor, reduce a noise of the compressor, and/or improve a longevity of the compressor.

Opposing outlets may also face in opposite directions to each other. The compressor may thereby pass air in opposite directions therefrom. This may allow air to be passed with greater ease in opposite directions relative to the compressor in an air purifier comprising the compressor. For example, air may be passed from the compressor to locations in such an air purifier that are on opposite sides of the air purifier, relative to the compressor, while reducing a number of times, or an extent to which, the flow is redirected. This may improve an efficiency of the compressor and/or air purifier, such as by reducing restrictions to flow in the air purifier. Moreover, air may be passed to such locations in the air purifier without employing plural compressors located on opposite sides of the air purifier. This may reduce a cost, weight and/or complexity of such an air purifier comprising the compressor.

Optionally, the volute comprises a plurality of scroll passages extending circumferentially around the compressor, each scroll passage configured to pass air from the impeller to a respective one of the plurality of outlets. An airflow provided to each outlet through a respective scroll passage may be substantially independent of airflow through another scroll passage. In other words, an influence of an airflow to one outlet, via a respective scroll passage, on an airflow to another outlet, may be reduced, particularly when compared to a compressor in which airflow is provided to plural outlets via a single flow passage. This may improve a quality and/or distribution of air through each outlet.

Optionally, the scroll passages extend circumferentially around the axis of rotation of the impeller. Optionally, the scroll passages progressively increase in cross-sectional area, such as by progressively widening in one or more dimensions, around the compressor. This may cause a reduction in velocity and increase in pressure of the air passed from the impeller to each outlet of the plurality of outlets.

The scroll passages may be equally sized, so as to provide an equal flow rate and/or pressure to each outlet of the plurality of outlets. The cross-sectional area may be a cross-sectional area orthogonal to a direction of extension of the scroll passages. The scroll passages may widen in any suitable dimension. For instance, a radius of each scroll passage may increase, and/or a dimension of the scroll passage parallel to, or radial to, an axis of rotation of the impeller may increase, in a direction of extension of the scroll passage about the compressor.

Optionally, each scroll passage comprises a respective volute tongue, which defines at least a part of an outer wall of the scroll passage. Optionally, an outer wall of each scroll passage extends circumferentially about the axis of rotation from its respective volute tongue. Optionally, a distance of the outer wall of each scroll passage from the axis of rotation of the impeller increases progressively as the respective scroll passage extends around the axis of rotation of the impeller. Optionally, the volute tongues of each scroll passage are equally radially spaced from the axis of rotation of the impeller. In this way, the scroll passages may be substantially equally spaced around the axis of rotation, which may reduce cyclic loads on the impeller, and thereby reduce a wear and/or vibration associated with rotation of the impeller.

Optionally, each scroll passage extends circumferentially around the compressor about an angle equal to that of each other scroll passage. In other words, the plurality of scroll passages may have equal arc lengths around the compressor, such as around the axis of rotation of the impeller. In this way, each scroll passage may receive substantially the same amount of air from the impeller as each other scroll passage, thereby to provide an even distribution of air through the plurality of outlets. This may also provide a more even distribution of pressure around the impeller, which may reduce loads, and particularly imbalanced loads, on the impeller.

Optionally, each scroll passage comprises a respective outlet passage extending substantially tangentially from the compressor. The outlet passages may extend substantially tangentially to a circle that surrounds and is axially aligned with the impeller. Optionally, the volute tongue of each scroll passage at least in part defines a shared wall between the scroll passage and a neighbouring scroll passage and/or the impeller. In other words, the scroll passages may radially overlap each other at respective volute tongues. Optionally, the scroll passages comprise such a shared wall and/or radially overlap about an arc extending from a respective volute tongue up to 5°, up to 10°, up to 25°, up to 45°, up to 90°, up to 180° or over 180° about the axis of rotation of the impeller. Alternatively, the scroll passages may not radially overlap each other. In such a case, a tip of each volute tongue may define a fork between its respective scroll passage and the outlet passage of a neighbouring scroll passage. In any case, the outer walls of respective scroll passages may define at least a part of an outer surface of the compressor.

Optionally, each outlet passage increases in cross-sectional area between its associated outlet and its associated exit aperture. In other words, the exit aperture associated with each outlet passage may have a greater cross-sectional area than the outlet associated with the same outlet passage. In this way a velocity of air passing through an outlet passage may be reduced, while a pressure of the air may be increased. A higher pressure may be beneficial in air purifiers, in which filters and other obstacles may provide a restriction to air flow.

Optionally, an area of each outlet is equal to the area of each other outlet of the compressor, and/or wherein an area of each exit aperture is equal to an area of each other exit aperture of the compressor. This may provide a more even distribution of pressure around the impeller, such as to reduce a vibration and/or wear of the impeller, as described above. Moreover, a flow rate of air through each outlet and/or exit aperture may be the same as a flow rate through each other outlet and/or exit aperture.

Optionally, the compressor comprises two scroll passages, each extending 180 degrees about the compressor. In this way, the compressor may comprise two outlets opening in opposite directions. This may allow two streams of air to be provided in opposite directions from the compressor, which may improve a versatility of the compressor compared, for example, to a compressor comprising a single scroll passage and/or a single outlet. Moreover, by providing two scroll passages extending 180 degrees about the compressor, airflow from the impeller may be more evenly distributed to scroll passages on either side of the impeller. This may reduce loads on and/or vibrations of the impeller.

The airflow provided through each outlet may be substantially half of the airflow that the impeller is able to generate. That is, the airflow through one of the outlets may be substantially the same as airflow through the other. Moreover, the airflow provided through each outlet may be substantially the same as the airflow provided through a compressor comprising a single outlet, while reducing a weight, form factor, noise and/or vibration of the compressor compared to two such single-outlet compressors.

Optionally, the compressor is a centrifugal or mixed-flow compressor. In this way, a size of the compressor, such as a size in an axial direction along an axis of rotation of the impeller, may be reduced when compared, for example, to an axial compressor. In particular, the inlet of the compressor, where provided, may be axially aligned with an axis of rotation of the impeller, and the one or more outlets may be configured to pass air radially, or partially axially (such as in a mixed-flow compressor), with respect to the inlet.

Moreover, by orientating the outlets so that air is passed radially, or substantially radially, such as partially axially, with respect to the inlet and/or the axis of rotation of the impeller, the air may be directed from the compressor to different parts of an air purifier comprising the compressor with minimal further changes to a direction of flow from the compressor. For instance, fewer bends may be required in channels of such an air purifier, compared to an air purifier comprising a compressor with a single outlet. This may improve an ease with which the air can flow through such channels, and thereby reduce a pressure required to be provided by the compressor. This may, in turn, reduce a speed, power draw, and/or noise of the compressor.

Optionally, the compressor is configured to operate with a power draw of no greater than 15 W, in use. Optionally, the compressor is configured to operate with a power draw of no greater than 12 W, 10 W, or 8 W in use. This may lead to a reduced power draw of an air purifier comprising the compressor. For instance, the compressor may be able to generate an airflow at a first flow rate through each of N outlets at a reduced power draw compared to a combined power draw of N compressors generating an airflow at the first flow rate through respective single outlets. This may be particularly beneficial when the air purifier is battery-operated.

Optionally, the compressor is configured to supply air to the plurality of outlets at a combined flow rate not exceeding 12 l/s. That is, when the compressor comprises two outlets, the compressor is configured to supply air to each outlet at a flow rate not exceeding 6 l/s. Optionally, the compressor is configured to supply air to the plurality of outlets at a combined flow rate not exceeding 10 l/s, 8 l/s or 6 l/s. Such flow rates may allow an air purifier, and in particular a head-wearable air purifier, comprising the compressor to be used when outside, and/or when exercising. For instance, for a head-wearable air purifier, the flow rate may be such that a majority of the filtered airflow is provided to a user's face when a breathing rate of the user is increased, and/or in the presence of external influences, such as wind.

Optionally, the compressor is configured to supply air to the plurality of outlets at a rotational speed of the impeller not exceeding 20,000 rpm. Optionally, the compressor is configured to supply air to the plurality of outlets at a rotational speed of the impeller not exceeding 17,000 rpm, 15,000 rpm, 13,000 rpm, or 12,000 rpm. This may lead to reduced noise of the compressor, and/or a reduced battery draw of an air purifier comprising the compressor. The rotational speed of the impeller may be such that a suitable pressure and/or flow rate is produced to allow a user to use a head-wearable air purifier comprising the compressor when outside and/or when exercising.

Optionally, an area of the compressor, when viewed in an axial direction along an axis of rotation of the impeller, is no greater than 150 mm². Optionally, the area is no greater than 130 mm², 110 mm², 100 mm², or 90 mm². Optionally, a depth of the compressor, in the axial direction, is no greater than 50 mm, 40 mm, 30 mm, 20 mm, or 10 mm. In this way, a form factor of the compressor may be such that the compressor, when comprised in a head-mountable air purifier, is more comfortable when worn on a user's head. This may also reduce an extent of protrusion of the compressor from the user's head, reducing a likelihood of the compressor, or a housing of the head-wearable air purifier surround the compressor, catching on external objects.

Moreover, a weight of the compressor may be reduced compared, for example, to a combined weight of plural single-outlet compressors that can generate an airflow at the same, or a similar, flow rate through the single outlet as the compressor can generate through each of the plurality of outlets. This may further improve a comfort of a head-wearable air purifier comprising the compressor.

A second aspect of the present invention provides a purifier comprising a filter and the compressor of the first aspect for causing air to flow through the filter to generate a filtered airflow. Advantageously, by providing a compressor comprising plural outlets, air may be passed from the compressor in plural different directions in the air purifier. For instance, a single compressor may provide plural airstreams in the air purifier. In this way, a number of compressors in the air purifier may be reduced compared, for example, to an air purifier comprising two compressors each comprising only one outlet. This may reduce a weight, cost and/or noise of the compressor and/or an air purifier comprising the compressor. Fewer compressors may lead to fewer potential points of wear, or failure, and thereby a more reliable air purifier. Moreover, it may be easier and/or cheaper to repair an air purifier having fewer compressors.

Further advantageously, the air purifier may require fewer components than, for example, an air purifier comprising plural single-outlet compressors for passing air to respective different parts of the air purifier. For instance, as air may be expelled from the compressor in different directions therefrom, fewer bends for redirecting the flow in the head-wearable air purifier may be required. This may result in a lighter, simpler and cheaper air purifier.

The filter may be located upstream of the compressor, such as upstream of an inlet of the compressor. Alternatively, the filter may be located downstream of one or more of the plurality of outlets. In either case, the filter may be a part of the compressor, or may be external to the compressor. Further alternatively, the filter may be located in the compressor, such as between the inlet and the impeller, or between the impeller and one or more of the outlets. By locating the filter upstream of the inlet, where provided, the compressor may be kept clear of debris. In such a case, the compressor may be configured to provide the filtered airflow from each of the plurality of outlets. Alternatively, when the filter is downstream of the compressor, the airflow generated by the compressor may flow through the filter to provide the filtered airflow downstream of the filter.

It will be appreciated that the air purifier may benefit from any of the optional features and/or advantages mentioned above in relation to the compressor of the first aspect.

Optionally, the air purifier is a head-wearable air purifier. It will be appreciated that such a head-wearable air purifier may benefit from any of the optional features and/or advantages mentioned above in relation to the compressor of the first aspect and/or the air purifier of the second aspect.

For instance, the compressor may provide the same or similar airflow as two single-outlet compressors while being more lightweight and/or experiencing reduced vibrations, noise and/or power draw, which may be particularly advantageous on a head-wearable air purifier.

Optionally, the head-wearable air purifier comprises a nozzle assembly for passing air received from one or more of the plurality of outlets to a user. Optionally, the nozzle assembly provides the filtered airflow to the user. Optionally, the nozzle assembly provides the air, such as the filtered airflow, to the user's face. The nozzle assembly may be configured such that, in use, with the head wearable air purifier located on a head of a wearer, the nozzle assembly extends in front of a face of the wearer, for example such that an air outlet of the nozzle assembly is located in a region of a mouth and/or lower nasal region of the wearer. In other words, the nozzle assembly may be located at a “front” of the head-wearable air purifier. In particular, the nozzle assembly may be located fore of a coronal plane of the head-wearable air purifier, wherein the coronal plane is intended to be substantially aligned with a coronal plane of a wearer's body, in use.

The nozzle assembly may be configured such that, in use, the nozzle assembly extends in front of a face of the wearer without contacting the face of the wearer. This may provide an arrangement with increased comfort for the wearer, for example relative to an arrangement where the nozzle assembly contacts a face of a wearer in use. The nozzle assembly may be generally elongate and arcuate in form. The air outlet may be substantially centrally located along the nozzle assembly.

Optionally, the nozzle assembly comprises a first end section on a first side of the head-wearable air purifier, and a second end section on a second side of the head-wearable air purifier, the second side opposite to the first side. The nozzle assembly may comprise first and second apertures in the respective first and second end sections for receiving airflow from the compressor. In particular, the first aperture may be configured to receive airflow from a first outlet of the compressor, and the second aperture may be configured to receive airflow from a second outlet of the compressor. In this way, the compressor comprising plural outlets presents a convenient way of delivering airflow to opposing sides of the nozzle assembly.

The airflow received through the first and second apertures may be combined in the nozzle assembly to provide a combined airflow to a user's face. The airflow from the first aperture may collide with the airflow from the second aperture. By combining the airflow from the first and second apertures, the combined airflow may be presented to a user's face more symmetrically than, for example, an airflow received from just one side of the nozzle assembly.

Optionally, the compressor is located at an opposite side of the air purifier to the nozzle assembly. Optionally, when the nozzle assembly is located fore of a coronal plane of the head-wearable air purifier, the compressor may be located aft of the coronal plane. In other words, the compressor may be located to a rear of a wearer's head, in use.

Optionally, the head-wearable air purifier comprises a median sagittal plane. Optionally, the median sagittal plane is intended to be aligned with a median sagittal plane of a wearer's body, when worn. In this way, the median sagittal plane is orthogonal to the coronal plane. Optionally, the nozzle assembly intersects the median sagittal plane. Optionally, a centre of the nozzle assembly is aligned with the median sagittal plane. Optionally, the compressor is located so as to intersect the median sagittal plane. Optionally, a centre of the compressor, such as a point within the compressor located on an axis of rotation of the impeller, is aligned with the median sagittal plane. Optionally, the centre of the compressor is located so that an angle between the median sagittal plane and a line connecting the centre of the compressor and an intersection between the coronal and median sagittal planes is up to 1, up to 5, up to 10, up to 20, up to 30, up to 50, or up to 70 degrees.

In this way, the compressor may be distanced from the ears of a wearer, in use. As such, a level of noise of the compressor detectable by a wearer may be reduced. This may also allow the head-wearable air purifier to be wearable absent a supporting structure, or device, such as a speaker assembly, covering the wearer's ear(s). This may improve a versatility of the head-wearable air purifier. For example, the wearer may be able to better hear their surroundings, and so may wear the head-wearable air purifier during activities requiring increased awareness, such during sport or other exercise, in an emergency situation, or when communicating with other people. For instance, the head-wearable air purifier may be a helmet, such as a cycle or motorcycle helmet, or a firefighter's helmet.

Alternatively, the compressor may be located at a top of the head-wearable air purifier, which is located at the top of a user's head, in use. For instance, the compressor may be located on a side of the compressor which is substantially orthogonal to both the coronal and median sagittal planes of the head-wearable air purifier. This may improve a stability of the head-wearable air purifier, such as by moving a centre of gravity of the purifier towards a centre of a wearer's body when viewed from above.

Optionally, the head-wearable air purifier is absent any compressor at one or both lateral sides of the head mounted purifier, the lateral sides being located at opposite lateral sides of the wearer's head, in use. This may reduce a lateral weight of the head-wearable air purifier. This, in turn, may improve a balance and/or comfort of the head-wearable air purifier.

Optionally, the head-wearable air purifier comprises at least two channels configured to pass compressed air along opposing sides of the head-wearable air purifier from a respective outlet of the plurality of outlets.

Optionally, the at least two channels pass compressed air to the nozzle assembly, where provided. The opposite sides may be at opposite sides of the medial sagittal plane. As such, the opposing sides may be located at opposite sides of a wearer's head, in use. In this way, the compressed air from the compressor may be conveniently delivered to opposing sides of the nozzle assembly via the respective at least two channels. Moreover, by providing channels on opposing sides of the head-wearable air purifier, the head-wearable air purifier may be more symmetrical, and so may be more stable when worn.

The at least two channels may comprise a first channel for passing air from a first outlet of the compressor to the first aperture of the nozzle assembly, and a second channel for passing air from a second outlet of the compressor to the second aperture of the nozzle assembly, where provided. The first and second end portions of the nozzle assembly, and so also the first and second apertures, may be located on opposite sides of the median sagittal plane.

Optionally, the air purifier comprises a single compressor for generating the filtered airflow. In other words, the compressor may be the sole compressor of the head-wearable air purifier for generating the filtered airflow. Optionally, the head-wearable air purifier is absent any other compressor, such as a compressor for generating an airflow other than the filtered airflow. By the compressor being the sole compressor of the head-wearable air purifier for generating the filtered airflow, the compressor may be lighter, quieter, simpler, cheaper, and/or easier to maintain than a head-wearable air purifier comprising more than one compressor, such as more than one compressor for generating the filtered airflow, for any of the reasons stated above in relation to the head-wearable air purifier comprising fewer compressors and/or impellers.

Moreover, by providing only a single compressor for generating the filtered airflow, there may be no need to synchronise, for example, a speed of, or pressure or flow rate generated by, plural compressors for generating filtered airflow. This may, in turn, lead to a simpler control system for controlling the compressors and/or the head-wearable air purifier as a whole.

Optionally, the head-wearable air purifier is battery-powered. Optionally, the head-wearable air purifier comprises a battery. In this way, the head-wearable air purifier may be portable, and in particular may be used away from any external power source.

Optionally, the battery is rechargeable. Optionally, the battery is replaceable. This may allow the head-wearable purifying device to be used more regularly, such as without waiting for a depleted battery to be re-charged. For instance, a depleted battery could be replaced with a charged, or at least partially charged battery. The depleted battery could then be re-charged separately.

### BRIEF DESCRIPTION OF THE DRAWINGS

Examples of the invention will be described with reference to the Figures in which:

FIG. 1 shows an isometric schematic view of a compressor;

FIG. 2 shows a cross-sectional view through the compressor in the direction A-A shown in FIG. 1;

FIG. 3 shows a cross-sectional view through the compressor 1 in the direction B-B shown in FIG. 1, with an impeller of the compressor shown in its entirety, for clarity;

FIG. 4 shows an isometric schematic view of the compressor as shown in FIG. 3; and

FIGS. 5A and 5B respectively show a side-profile and top-down view of a head-wearable air purifier comprising the compressor shown in FIGS. 1 to 4.

### DETAILED DESCRIPTION OF THE INVENTION

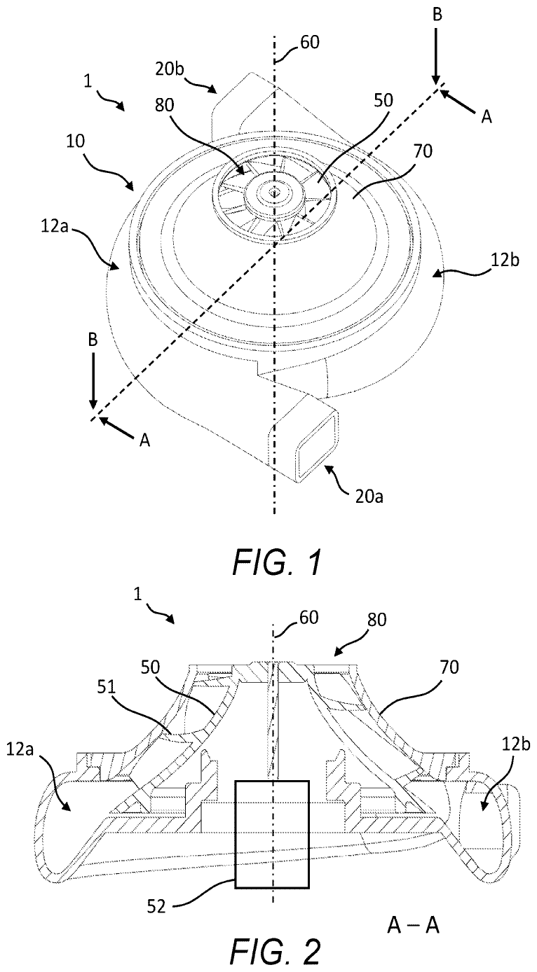

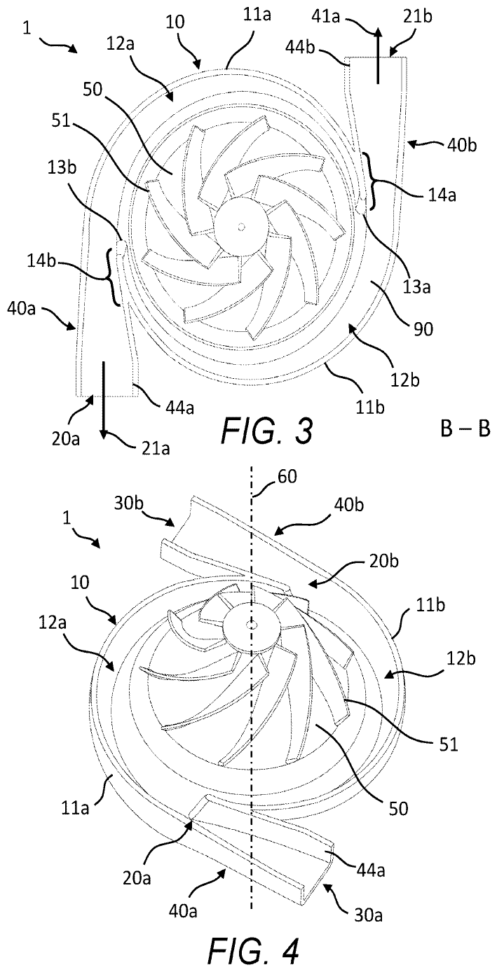

FIGS. 1 to 4 show a compressor 10 comprising an impeller 50, two outlets 20a, 20b, and a volute 10 that is configured to provide air from the impeller 50 to each of the two outlets 20a, 20b. The compressor 1 also comprises, as best shown in FIGS. 3 and 4, a cover 70 that covers the impeller 50, and is axially aligned with an axis of rotation 60 of the impeller 50. The cover 70 also defines an inlet 80 to the impeller 50. The cover 70 protects the impeller 50 from external debris, and also helps to guide fluid from the inlet 80, through the impeller 50, towards the volute 10.

The impeller 50 is here a mixed-flow impeller 50, so that air enters the compressor 1 and the impeller 50, through the inlet 80 in a substantially axial direction along the axis of rotation 60, and leaves the impeller 50 and/or the compressor 1 in a direction that is oblique to a plane orthogonal to the axis of rotation 60. In particular, the airflow leaves the impeller 50 in a mostly radial, but partly axial, direction. It will be appreciated that, in other examples, the compressor 1 may be a centrifugal compressor, so that air enters the compressor 1 in the axial direction and leaves the impeller 50 and/or the compressor 1 in a substantially radial direction, orthogonal to the axis of rotation 60.

The compressor 1, and in particular the impeller 50, is configured to impart energy to, and increase a pressure of, air passing through the compressor 1. The impeller 50 of the present example is absent any splitter blades. That is, the blades 51 of the impeller are substantially the same shape and size, and are distributed evenly around a circumference of the impeller 50. The compressor 1 is also absent any diffuser vanes surrounding the impeller 50. It will be appreciated that, in other examples, the impeller 50 may comprise splitter blades and/or the compressor 1 may comprise diffuser vanes surrounding the impeller 50.

The volute 10 comprises two progressively widening scroll passages 12a, 12b, each extending 180° about the compressor 50, specifically about the axis of rotation 60 of the impeller 50. The scroll passages 12a, 12b progressively increase in cross-sectional area as they extend about the axis of rotation 60 towards the respective outlets 20a, 20b. The cross-sectional area is here a cross-sectional area in a radial plane comprising the axis of rotation 60. Each scroll passage 12a, 12b is open to the impeller 50 about the full 180° of extension. The scroll passages 12a, 12b are thereby configured to receive airflow that has been accelerated by the impeller 50, and to guide the received airflow to the respective outlets 20a, 20b. In the present example, the outlets 20a, 20b are a part of the respective scroll passages 12a, 12b.

Each scroll passage 12a, 12b comprises a respective volute tongue 13a, 13b, which defines at least a part of an outer wall 11a, 11b of the scroll passage 12a, 12b. In particular, an outer wall 11a, 11b of each scroll passage 12a, 12b starts from, and extends circumferentially about the axis of rotation 50 from, its respective volute tongue 13a, 13b. A distance of each outer wall 11a, 11b from the axis of rotation 50 increases progressively as each scroll passage extends around the axis of rotation 50. The volute tongues 13a, 13b of each scroll passage 12a, 12b are diametrically opposed in the compressor 10, and are equally radially spaced from the axis of rotation 60 of the impeller 50. A tongue length 14a, 14b of the volute tongue 13a, 13b of each scroll passage 12a, 12b radially delimits the other scroll passage 12a, 12b from the scroll passage 12a, 12b and the impeller 50. In this way, each scroll passage 12a, 12b is open to the impeller 50 about the axis of rotation 60, from its volute tongue 13a, 13b to the volute tongue 13a, 13b of the other scroll passage 12a, 12b.

Each scroll passage 12a, 12b in the present example comprises an outlet passage 40a, 40b which departs substantially tangentially from the compressor 1 to a respective outlet 20a, 20b. The outlet passages 40a, 40b are defined by a respective inner wall 44a, 44b and an outer wall 11a, 11b of a respective scroll passage 12a, 12b. In each case, the inner wall 44a, 44b is radially inward from the respective outer wall 11a, 11b. The inner wall 44a, 44b of the outlet passage 40a, 40b of each scroll passage 12a, 12b extends, from the volute tongue 13a, 13b of the other scroll passage 12a, 12b, away from the outer wall 11a, 11b of the other scroll passage 12a, 12b. In this way, the volute tongue 13a, 13b of each scroll passage 12a, 12b defines a point at which the outer wall 11a, 11b of the scroll passage 12a, 12b and the inner wall 44a, 44b of the outlet passage 40a, 40b of the other scroll passage 12a, 12b meet.

In this way, the tongue length 14a, 14b of the volute tongue 13a, 13b of each scroll passage 12a, 12b radially delimits the outlet passage 40a, 40b of the other scroll passage 12a, 12b from the scroll passage 12a, 12b and the impeller 50. That is, the volute tongue 13a, 13b of each scroll passage 12a, 12b at least in part defines a shared wall between the scroll passage 12a, 12b and the other scroll passage 12a, 12b. In other words, the scroll passages 12a, 12b radially overlap each other only at the tongue lengths 14a, 14b of the respective volute tongues 13a, 13b, which here is an overlap over a region extending about 25° around the axis of rotation 60. The outer walls 11a, 11b of respective scroll passages 12a, 12b otherwise define at least a part of an outer surface of the compressor 1.

Each outlet 20a, 20b here has the same shape and cross-sectional area as each other respective outlet 20a, 20b, but these may alternatively have different shapes and/or cross-sectional areas. A cross-sectional area in a direction of extension of each outlet passage 40a, 40b here increases progressively towards the respective outlets 20a, 20b. That is, each outlet 20a, 20b has an area, defined between an inner wall 44a, 44b of a respective outlet passage 40a, 40b and an outer wall 11a, 11b of a respective scroll passage 12a, 12b, that is larger than a cross-sectional area between a the outer wall 11a, 11b of the respective scroll passage 12a, 12b and the volute tongue 13a, 13b of the other scroll passage 12a, 12b. In other examples, these areas may be substantially the same size. That is, the outlet passages 40a, 40b may not increase in cross-sectional area in a direction of extension of the outlet passages 40a, 40b.

The compressor 1, as shown in FIG. 2, comprises a motor 52 for causing the impeller 50 to rotate about the axis of rotation 60. The motor 52 is here an electric motor, and specifically a brushless motor, but may be any other suitable motor for driving the impeller 50.

In use, power is supplied to the motor 52 to cause the impeller 50 to rotate within the cover 70. In the present example, the compressor 1, and in particular the motor 52, is configured to operate with a power draw of no greater than 15 W, in use. The compressor 1 is configured to supply air from the outlets 20a, 20b at a combined flow rate not exceeding 12 l/s. That is, the compressor 1 is configured to supply air to each outlet 20a, 20b at a flow rate not exceeding 6 l/s. The compressor 1 is configured to supply air to the outlets 20a, 20b at a rotational speed of the impeller 50 not exceeding 20,000 rpm. An area of the compressor 1, when viewed in an axial direction along the axis of rotation 60 of the impeller 50, is no greater than 150 mm², and a depth of the compressor, in the axial direction, is no greater than 50 mm.

The cover 70 is here so shaped, as best shown in FIGS. 1 and 2, to substantially conform to a shape of the impeller 50. In this way, when the impeller rotates 50, air is drawn substantially axially through the inlet 80 and guided between the impeller 50 and the cover 70. The impeller 50 accelerates the air outwardly from the axis of rotation 60, so that the air leaves the impeller 50 in a substantially radial, but partly axial, direction, as discussed above. The cover 70 is also so shaped that the impeller 50 is open to the volute 10, and in particular to the scroll passages 12a, 12b, around a circumference of the impeller 50. In this way, the airflow generated by the impeller 50 is expelled from the impeller 50 into the scroll passages 12a, 12b on either side of the impeller 50. The airflow is then directed around the scroll passages 12a, 12b into the respective outlet passages 40a, 40b and towards the respective outlets 20a, 20b. Because the scroll passages 12a, 12b progressively widen as they extend around the compressor 1, and further widen along the outlet passages 40a, 40b, a pressure of the airflow is increased as it flows towards the outlets 20a, 20b. The airflow generated by the impeller 50 is then expelled through the outlets 20a, 20b in the directions shown by the arrows labelled 21a and 21b in FIG. 3. That is, the air is expelled in opposite directions from the compressor 1. In this way, the compressor finds particular utility in scenarios where airflow in multiple directions is beneficial, such as in air purifiers, and in particular head-wearable air purifiers.

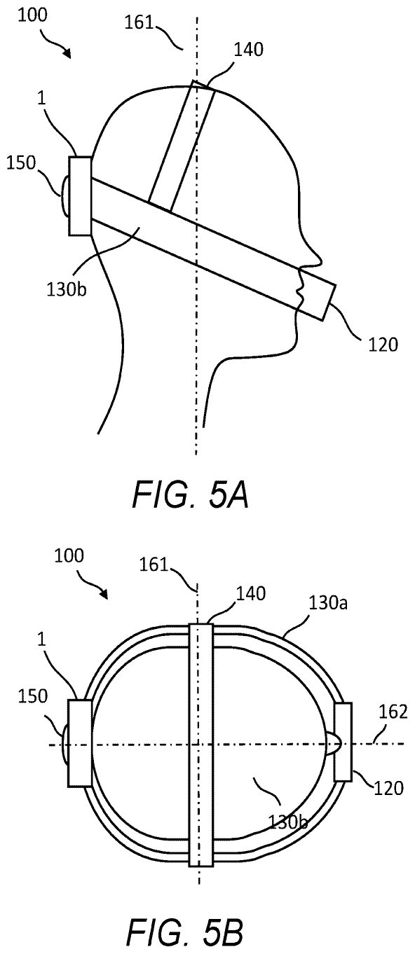

FIG. 5A shows a schematic side-view of such a head-wearable air purifier 100 (which may herein be simply referred to as a “purifier” or “air purifier”) when worn on a wearer's head. FIG. 5B shows a schematic top-down view of the same head-wearable air purifier. Configurations of the head-wearable air purifier 100 are here defined with reference to a coronal plane 161 of the head-wearable air purifier, which is intended to be substantially aligned with a coronal plane 161 of a wearer's body, in use. Configurations are also defined with reference to a median sagittal plane 162 which is orthogonal to the coronal plane 161 and which is intended to be substantially aligned with a median sagittal plane of the wearer's body, in use.

The air purifier comprises the compressor 1 for generating airflow through the air purifier, a nozzle assembly 120 positioned to supply airflow to a wearer's face, and two channels 130a, 130b for passing airflow from the respective exit apertures of the compressor 1 to the nozzle assembly 120. The air purifier 100 also comprises a structure 140 for mounting and/or locating the air purifier 100 on a wearer's head. Here, the structure 140 comprises a strap, but in other examples may be any other suitable structure. For instance, the structure 140 may comprise a main body of a hat or helmet. The air purifier 100 may, in other examples, comprise more than two channels 130a, 130b, or a single channel for passing air from both exit apertures 42a, 42b of the compressor 1 to the nozzle assembly 120.

The air purifier 100 comprises a filter 150, which is here located upstream of the compressor 1. In this way, the compressor 1 is configured to generate a filtered airflow in the air purifier 100. The filter 150 is specifically located upstream of the inlet 80 of the compressor 1, and is more specifically an annular filter located in an annular intake leading to the inlet 80 of the compressor. It will appreciated, however, that the filter 150 may be of any other suitable shape and/or may be located in any other suitable location in the air purifier 100. For instance, the filter 150 may be a substantially circular filter and/or may be located in a circular intake to the inlet 80 of the compressor 1. Alternatively, or in addition, the air purifier 100 may comprise a filter located downstream of the compressor 1, such as in one or both of the channels 130a, 130b and/or in the nozzle assembly 120.

As shown in FIGS. 5A and 5B, the nozzle assembly 120is configured such that, in use, with the head wearable air purifier located on a head of a wearer, the nozzle assembly 120 extends in front of a face of the wearer, specifically such that an air outlet of the nozzle assembly is located in a region of a mouth and/or lower nasal region of the wearer. In other words, the nozzle assembly 120 is located at a “front” of the head-wearable air purifier 100. In particular, the nozzle assembly 120 is located fore of the coronal plane 161 of the head-wearable air purifier 100. The compressor 1, in contrast, is located toward a rear of a wearer's head, in use. In particular, the compressor 1 is located on an opposite side of the head-wearable purifier to the nozzle assembly 120, specifically aft of the coronal plane 161. The compressor 1 and nozzle assembly 120 are here also centrally located on the median sagittal plane 162, so that they overlap the median sagittal plane 162. The exit apertures 42a, 42b of the compressor 1 are here orientated horizontally, so that they point towards opposite sides of the median sagittal plane 162. In this way, the compressor 1 can expel air to either side of the sagittal plane 162, and in particular into respective channels 130a, 130b of the head-wearable air purifier. The channels 130a, 130b then extend around the air purifier from the compressor 1 to the nozzle 120 on either side of the median sagittal plane 162.

It will be appreciated that the compressor 1, channels 130a, 130b and/or the nozzle assembly 120 can be arranged in any other suitable arrangement on the head-wearable air purifier 100, while still benefitting from the advantages described above. It will also be appreciated that, while the compressor 1 has here been described in relation to its implementation in a head-wearable air purifier 100, the compressor 1 may equally be employed in any other suitable air purifier, for example, a room purifier or a vehicle cabin purifier. Moreover, the head-wearable air purifier 100 may, in some examples, be a part of a hat or helmet, such as a motorbike helmet, bicycle helmet, and/or firefighter's helmet. The air purifier 100 may also comprise other components, such as a visor, speakers such as earphones, electronic controls, and/or any other suitable components as a skilled person may wish to employ a head-wearable air purifier 100.

It will further be appreciated that, while the compressor 1 has here been described as comprising two scroll passages 12a, 12b with corresponding outlets 20a, 20b, the compressor 1 may alternatively have more than two, such as 3, 4 or more than 4 scroll passages and respective outlets. In such examples, the scroll passages may be similarly evenly spaced around the compressor 1, and may or may not overlap neighbouring scroll passages. For instance, the compressor may comprise three scroll passages each extending 120°around the compressor 1, and three corresponding outlets equally circumferentially spaced at increments of 120° around the compressor 1. This may allow the compressor 1 to expel more than two, such as three or four streams of air in different directions therefrom. It will be appreciated that such a compressor 1, and/or a head-wearable air purifier 100 comprising such a compressor 1, may benefit from any of the optional features and advantages described above.

Other variations and modifications within the scope of the appended claims will be conceivable by the skilled person.

1. A compressor for an air purifier, the compressor comprising:
an impeller;
a plurality of outlets; and
a volute extending circumferentially around the compressor, the volute configured to pass air from the impeller to each of the plurality of outlets.

2. The compressor of claim 1, wherein the plurality of outlets are circumferentially spaced around the compressor.

3. The compressor of claim 1, wherein each outlet of the plurality of outlets is located on an opposite side of the compressor to another outlet of the plurality of outlets.

4. The compressor of claim 1, wherein the volute comprises a plurality of scroll passages extending circumferentially around the compressor, each scroll passage configured to pass air from the impeller to a respective one of the plurality of outlets.

5. The compressor of claim 4, wherein each scroll passage extends circumferentially around the compressor about an angle equal to that of each other scroll passage.

6. The compressor of claim 4, wherein each scroll passage comprises a respective outlet passage extending substantially tangentially from the compressor.

7. The compressor of claim 6, wherein each outlet passage increases in cross-sectional area between its associated outlet and its associated exit aperture.

8. The compressor of claim 7, wherein an area of each outlet is equal to the area of each other outlet of the compressor, and/or wherein an area of each exit aperture is equal to an area of each other exit aperture of the compressor.

9. The compressor of claim 4, wherein the compressor comprises two scroll passages, each extending 180 degrees about the compressor.

10. The compressor of claim 1, wherein the compressor is a centrifugal or mixed-flow compressor.

11. The compressor of claim 1, wherein the compressor is configured to operate with a power draw of no greater than 15 W, in use.

12. The compressor of claim 1, wherein the compressor is configured to supply air to the plurality of outlets at a combined flow rate not exceeding 12 l/s.

13. The compressor of claim 1, wherein the compressor is configured to supply air to the plurality of outlets at a rotational speed of the impeller not exceeding 20,000 rpm.

14. The compressor of claim 1, wherein an area of the compressor, when viewed in an axial direction along an axis of rotation of the impeller, is no greater than 150 mm².

15. An air purifier comprising a filter and the compressor of claim 1, the compressor configured to cause air to flow through the filter to generate a filtered airflow.

16. The air purifier according to claim 15, wherein the air purifier is a head-wearable air purifier.

17. The air purifier of claim 16, further comprising a nozzle assembly for passing air received from one or more of the plurality of outlets to a user.

18. The air purifier of claim 17, wherein the compressor is located at an opposite side of the air purifier to the nozzle assembly.

19. The air purifier of claim 17, wherein the air purifier comprises a single compressor for generating the filtered airflow.

20. The air purifier of claim 17, wherein the head-wearable air purifier is battery-powered.
